(19) United States
(12) Patent Application Publication
(10) Pub. No.: US 2024/0408327 A1
(43) Pub. Date: Dec. 12, 2024

(54) INTRAORAL AEROSOL DELIVERY DEVICE

(71) Applicant: Oron ZACHAR, Tel Aviv (IL)

(72) Inventor: Oron ZACHAR, Tel Aviv (IL)

(21) Appl. No.: 18/701,234

(22) PCT Filed: Oct. 20, 2021
(86) PCT No.: PCT/IB2021/000724

§ 371 Date: Apr. 14, 2024

(51) Int. Cl. A61M 15/00

(52) U.S. Cl. CPC A61M 15/0085; A61M 15/001; A61M 2205/0294; A61M 2205/3334; A61M 2205/3344; A61M 2210/065

(57) ABSTRACT

An electrically-powered inhalation device for delivery of an aerosol to the oropharynx of a user includes a distal portion housing a piezo assembly including an ultrasonically vibrable mesh membrane, and a neck portion including a narrow section characterized by a minimum cross-sectional dimension that is at least 10% smaller than a minimum cross-sectional dimension of the distal portion passing through and parallel to the mesh membrane. At least a part of the narrow section is displaced proximally from the mesh membrane by at least 0.5 cm and not more than 6 cm. The inhalation device is shaped such that when the user's lips and/or teeth are transversely engaged with the narrow section, the mist-generating location resides distal to the user's teeth within the user's oral cavity and the mist-exiting location is in direct fluid communication with the user's oropharynx.

### CROSS-REFERENCE TO RELATED APPLICATIONS

The following patent applications are incorporated herein by reference in their entirety: PCT/IB2021/053088 filed on Apr. 14, 2021, U.S. Provisional Patent Application No. 62/897,340 filed on Sep. 8, 2019, and U.S. Provisional Patent Application No. 62/993,884 filed on Mar. 24, 2020, both of which are incorporated herein by reference in their entirety.

### FIELD OF THE INVENTION

The present invention relates to mist-delivery devices and refillable and/or replaceable containers for use therein, and to methods for using such devices. In particular the present invention relates to devices for intraoral use for delivering an aerosol to a user's oropharynx.

### BACKGROUND

Existing oral inhalers suffer from the problem that any mist produced must traverse the tongue and other parts of the oral cavity, causing part of any dosed substance to fail to reach the oropharynx. Therefore a need exists for an intraoral inhaler capable of delivering a precise dosage of a substance to a user's oropharynx, preferably configured to place a mist-generating location and/or mist-exiting location of the inhaler far enough into the oral cavity to overcome the aforementioned shortcoming. There is also a need for such an inhalation device to be compact and comfortable to use.

### SUMMARY

According to embodiments disclosed herein, an electrically-powered inhalation device for delivery of an aerosol to the oropharynx of a user comprises: (a) respective proximal and distal portions, the proximal portion including an inlet for a liquid, the distal portion including (i) an aerosol outlet defining a mist-exiting location and (ii) a piezo assembly including an ultrasonically vibrable mesh membrane, for producing, upon electrical activation, a mist comprising droplets of the liquid, the mesh membrane defining a mist-generating location; and (b) an intermediate portion disposed distally from the proximal portion and proximally from the distal portion, wherein the inhalation device is shaped such that when the user's lips and/or teeth are transversely engaged with the intermediate portion, the mist-generating location resides within the user's oral cavity and the mist-exiting location is in direct fluid communication with the user's oropharynx.

According to embodiments, an electrically-powered inhalation device for delivery of an aerosol to the oropharynx of a user comprises: (a) respective proximal and distal portions, the proximal portion including an inlet for a liquid, the distal portion including (i) an aerosol outlet defining a mist-exiting location and (ii) a piezo assembly including an ultrasonically vibrable mesh membrane, for producing, upon electrical activation, a mist comprising droplets of the liquid, the mesh membrane defining a mist-generating location; and (b) an intermediate portion disposed distally from the proximal portion and proximally from the distal portion, wherein the distal portion is dimensioned to vertically span the user's oral cavity from tongue to hard-palate when the user's lips and/or teeth are transversely engaged with the intermediate portion, so as to place the mist-exiting location in fluid communication with the user's oropharynx.

In some embodiments, the liquid-inlet can be configured to receive liquid from a container, the liquid-inlet and the container having respective mating arrangements for mating with each other. In some embodiments, the mating can be reversible.

In some embodiments, the container can be detachably attachable to the proximal portion. In some embodiments, the inhalation device can additionally comprise the container. In some embodiments, the proximal portion can comprise a compartment for storing the liquid.

In some embodiments, the inhalation device can additionally comprise a portable power source. In some embodiments, the inhalation device can additionally comprise an inhalation sensor for monitoring a flow in an inhalation flow-path. In some embodiments, the inhalation sensor can be effective to detect an air pressure in the inhalation-flow path. In some embodiments, the inhalation sensor can be effective to detect a difference between an air pressure in the inhalation flow-path and an ambient air pressure outside the inhalation device. In some embodiments, the inhalation device can comprise control circuitry configured to initiate and/or cease activation of the mesh membrane in response to a result of the monitoring of the flow in the inhalation-path.

In some embodiments, the inhalation device can additionally comprise an exhalation sensor for monitoring a flow in an exhalation-flow path. In some embodiments, the exhalation sensor can be configured to detect a concentration of a chemical compound in the exhalation-flow path. In some embodiments, the chemical compound can be a component of the liquid. In some embodiments, the inhalation device can comprise control circuitry configured to cease or delay activation of the mesh membrane in response to a result of the monitoring of the flow in the exhalation flow path.

In some embodiments, the mesh membrane can be effective to eject at least 5 times, or at least 10 times, or at least 20 times, or at least 50 times more liquid in the mist during user inhalation than during user exhalation.

In some embodiments, the inhalation device can comprise an inhalation flow-path and an exhalation flow-path, each of the flow-paths including a respective one-way fluid valve.

In some embodiments, at least a portion of the distal portion can comprise a coating for generating a taste and/or odor sensation. In some embodiments, at least a portion of the intermediate portion can comprise a coating for generating a taste and/or odor sensation.

In some embodiments, the inhalation device comprises control circuitry programmable to cause the mesh membrane to eject, in the mist, a liquid quantity that is either predetermined or received in an input from a user.

In some embodiments, at least a portion of the container can be above a plane longitudinally bisecting the intermediate portion when the device is rotated such that the plane is horizontal. In some embodiments, all of the container can be above a plane longitudinally bisecting the intermediate portion when the device is rotated such that the plane is horizontal.

In some embodiments, the distal portion can comprise a liquid-retaining compartment in fluid communication with the liquid inlet via a conduit, and the liquid-retaining compartment can be shaped to receive a quantity of the liquid via the conduit by force of gravity when the inhalation device is in a first orientation, and to retain at least a part of the quantity against the force of gravity when the inhalation device is in a second orientation. In some embodiments, the retaining can be by a wall of the liquid-retaining compartment, and wall can be effective to partially block an egress of the retained at least a part of the quantity.

In some embodiments, the second orientation can be such that substantially all of the mesh membrane is in liquid communication with the retained at least a part of the quantity. In some embodiments, the second orientation can be such that a surface liquid level in the liquid-retaining compartment is higher than a surface liquid level in the container.

In some embodiments, a maximum retainable fluid capacity of the liquid-retaining compartment is at least 0.5 cc and not more than 4 cc, or at least 1 cc and not more 3 cc, or at least 1.5 cc and not more 2.5 cc.

In some embodiments, a ratio of (i) a combined fluid capacity of the container and the conduit to (ii) a maximum retainable fluid capacity of the liquid-retaining compartment, can be at least 1 and not more than 4, or at least 1.5 and not more than 3, or at least 1.75 and not more than 2.5.

In some embodiments, the inhalation device can additionally comprise a capillary pathway for conveying a portion of the liquid by capillary action from the liquid-inlet to the mesh membrane or to within 1 mm of the mesh membrane.

In some embodiments, the mist-generating location can be at least 20% deep or at least 30% deep or at least 40% deep or at least 50% deep or at least 60% deep or at least 70% deep or at least 80% deep into an oral-cavity volume beneath the user's hard palate.

In some embodiments, the inhalation device can additionally comprise a display device configured to display information about at least one of: (i) a currently-remaining quantity of the liquid or of a component thereof, (ii) an already-misted quantity of the liquid or of a component thereof, and/or (iii) the identity of a component of the liquid.

In some embodiments in which the inhalation device includes an exhalation sensor, the inhalation device can additionally comprising a display device configured to display information about at least one of: (i) a currently-remaining quantity of the liquid or of a component thereof, (ii) an already-misted quantity of the liquid or of a component thereof, (iii) the identity of a component of the liquid, and (iv) the detected concentration of the chemical compound in the exhalation-flow path.

According to embodiments disclosed herein, an electrically-powered inhalation device for delivery of an aerosol to the oropharynx of a user comprises: (a) respective proximal and distal portions, the distal portion including (i) a volume for storing a liquid, (ii) an aerosol outlet defining a mist-exiting location and (iii) a piezo assembly including an ultrasonically vibrable mesh membrane, for producing, upon electrical activation, a mist comprising droplets of the liquid, the mesh membrane defining a mist-generating location; and (b) an intermediate portion disposed distally from the proximal portion and proximally from the distal portion, wherein the inhalation device is shaped such that when the user's lips and/or teeth are transversely engaged with the intermediate portion, the mist-generating location resides within the user's oral cavity and the mist-exiting location is in direct fluid communication with the user's oropharynx.

According to embodiments, an electrically-powered inhalation device for delivery of an aerosol to the oropharynx of a user comprises: (a) respective proximal and distal portions, the distal portion including (i) a volume for storing a liquid, (ii) an aerosol outlet defining a mist-exiting location and (iii) a piezo assembly including an ultrasonically vibrable mesh membrane, for producing, upon electrical activation, a mist comprising droplets of the liquid, the mesh membrane defining a mist-generating location; and (b) an intermediate portion disposed distally from the proximal portion and proximally from the distal portion, wherein the distal portion is dimensioned to vertically span the user's oral cavity from tongue to hard-palate when the user's lips and/or teeth are transversely engaged with the intermediate portion, so as to place the mist-exiting location in fluid communication with the user's oropharynx.

In some embodiments, the inhalation device can additionally comprise a portable power source. In some embodiments, the inhalation device can additionally comprise an inhalation sensor for monitoring a flow in an inhalation flow-path. In some embodiments, the inhalation sensor can be effective to detect an air pressure in the inhalation-flow path. In some embodiments, the inhalation sensor can be effective to detect a difference between an air pressure in the inhalation flow-path and an ambient air pressure outside the inhalation device. In some embodiments, the inhalation device can comprise control circuitry configured to initiate and/or cease activation of the mesh membrane in response to a result of the monitoring of the flow in the inhalation-path.

In some embodiments, the inhalation device can additionally comprise an exhalation sensor for monitoring a flow in an exhalation-flow path. In some embodiments, the exhalation sensor can be configured to detect a concentration of a chemical compound in the exhalation-flow path. In some embodiments, the chemical compound can be a component of the liquid. In some embodiments, the inhalation device can comprise control circuitry configured to cease or delay activation of the mesh membrane in response to a result of the monitoring of the flow in the exhalation flow path.

In some embodiments, the mesh membrane can be effective to eject at least 5 times, or at least 10 times, or at least 20 times, or at least 50 times more liquid in the mist during user inhalation than during user exhalation. In some embodiments, the inhalation device can comprise an inhalation flow-path and an exhalation flow-path, each of the flow-paths including a respective one-way fluid valve.

In some embodiments, at least a portion of the distal portion can comprise a coating for generating a taste and/or odor sensation. In some embodiments, at least a portion of the intermediate portion can comprise a coating for generating a taste and/or odor sensation.

In some embodiments, the inhalation device comprises control circuitry programmable to cause the mesh membrane to eject, in the mist, a liquid quantity that is either predetermined or received in an input from a user.

In some embodiments, a maximum retainable fluid capacity of the liquid-retaining compartment is at least 0.5 cc and not more than 4 cc, or at least 1 cc and not more 3 cc, or at least 1.5 cc and not more 2.5 cc.

In some embodiments, a ratio of (i) a combined fluid capacity of the container and the conduit to (ii) a maximum retainable fluid capacity of the liquid-retaining compartment, can be at least 1 and not more than 4, or at least 1.5 and not more than 3, or at least 1.75 and not more than 2.5.

In some embodiments, the inhalation device can additionally comprise a capillary pathway for conveying a portion of the liquid by capillary action from the liquid-inlet to the mesh membrane or to within 1 mm of the mesh membrane.

In some embodiments, the inhalation device can additionally comprise a capillary pathway for conveying a portion of the liquid by capillary action from within the liquid-storing volume to the mesh membrane

In some embodiments, the mist-generating location can be at least 20% deep or at least 30% deep or at least 40% deep or at least 50% deep or at least 60% deep or at least 70% deep or at least 80% deep into an oral-cavity volume beneath the user's hard palate.

In some embodiments, the inhalation device can additionally comprise a display device configured to display information about at least one of: (i) a currently-remaining quantity of the liquid or of a component thereof, (ii) an already-misted quantity of the liquid or of a component thereof, and/or (iii) the identity of a component of the liquid.

In some embodiments in which the inhalation device includes an exhalation sensor, the inhalation device can additionally comprise a display device configured to display information about at least one of: (i) a currently-remaining quantity of the liquid or of a component thereof, (ii) an already-misted quantity of the liquid or of a component thereof, (iii) the identity of a component of the liquid, and (iv) the detected concentration of the chemical compound in the exhalation-flow path.

According to embodiments disclosed herein, an electrically-powered inhalation device for delivery of an aerosol to the oropharynx of a user comprises: (a) a distal portion including (i) an aerosol outlet defining a mist-exiting location and (ii) a piezo assembly including an ultrasonically vibrable mesh membrane, for producing, upon electrical activation, a mist comprising droplets of the liquid, the mesh membrane defining a mist-generating location; and (b) a neck portion including a narrow section, the narrow section being characterized by a minimum cross-sectional dimension that is at least 10% smaller than a minimum cross-sectional dimension of the distal portion passing through and parallel to the mesh membrane, at least a part of the narrow section being displaced proximally from the mesh membrane by at least 0.5 cm and not more than 6 cm, wherein the inhalation device is shaped such that when the user's lips and/or teeth are transversely engaged with the narrow section, the mist-generating location resides distal to the user's teeth within the user's oral cavity and the mist-exiting location is in direct fluid communication with the user's oropharynx.

In some embodiments, the distal portion can comprise a distal casing encompassing the mesh membrane at least circumferentially.

In some embodiments, the at least a part of the narrow section can be displaced proximally from the mesh membrane by at least 0.5 cm and not more than 5.5 cm, or by at least 0.5 cm and not more than 5 cm, or by at least 0.5 cm and not more than 4.5 cm, or by at least 0.5 cm and not more than 4 cm, or by at least 1 cm and not more than 6 cm, or by at least 1 cm and not more than 5.5 cm, or by at least 1 cm and not more than 5 cm, or by at least 1 cm and not more than 4.5 cm, or by at least 1 cm and not more than 4 cm.

In some embodiments, the narrow section can be characterized by a minimum cross-sectional dimension that is at least 20% smaller than the minimum cross-sectional dimension of the distal portion passing through and parallel to the mesh membrane, or at least 30% smaller than the minimum cross-sectional dimension of the distal portion passing through and parallel to the mesh membrane, or at least 40% smaller than the minimum cross-sectional dimension of the distal portion passing through and parallel to the mesh membrane, or at least 50% smaller than the minimum cross-sectional dimension of the distal portion passing through and parallel to the mesh membrane.

In some embodiments, the minimum cross-sectional dimension of the narrow section and the minimum cross-sectional dimension of the distal portion can define vectors that are coplanar, or within ±15° of being coplanar, or within ±30° of being coplanar, or within ±45° of being coplanar.

In some embodiments, the inhalation device of can comprise a proximal portion that includes a power source for powering the piezo assembly.

In some embodiments, the inhalation device can comprise a proximal portion that includes a liquid inlet.

In some embodiments, the inhalation device can comprise a first proximal portion that includes a liquid inlet and a second proximal portion that includes a power source for powering the piezo assembly.

In some embodiments, it can be that an outlet of the proximal portion that includes a liquid inlet is detachably attachable to the neck portion such that an interior volume of the proximal portion that includes a liquid inlet is arranged to be in fluid communication with an interior volume of the neck portion when a pressure-activated one-way valve is activated by pressure from the proximal portion that includes a liquid inlet.

In some embodiments, a center of gravity of the inhalation device can be is displaced proximally from a distal end of the narrow section when the inhalation device is in a liquid-empty state.

In some embodiments, the inhalation device of any preceding claim, additionally comprising an inhalation sensor for monitoring a flow in an inhalation flow-path. In some such embodiments, the inhalation sensor can be effective to detect an air pressure in the inhalation-flow path. In some embodiments, the inhalation sensor can be effective to detect a difference between an air pressure in the inhalation flow-path and an ambient air pressure outside the inhalation device. In some embodiments, the inhalation device can comprise control circuitry configured to initiate and/or cease activation of the mesh membrane in response to a result of the monitoring of the flow in the inhalation flow path.

In some embodiments, the distal portion can comprise a liquid-retaining compartment in fluid communication with the neck portion, the liquid-retaining compartment being shaped to receive a quantity of the liquid from the neck portion by force of gravity when the inhalation device is in a first orientation, and to retain at least a part of the quantity against the force of gravity when the inhalation device is in a second orientation. In some such embodiments, the retaining can be by a wall of the liquid-retaining compartment, the wall being effective to partially block an egress of the retained at least a part of the quantity. In some embodiments, the second orientation can be such that substantially all of the mesh membrane is in liquid communication with the retained at least a part of the quantity. In some embodiments, the second orientation can be such that a surface liquid level in the liquid-retaining compartment is higher than a surface liquid level in the container.

In some embodiments, the inhalation device can be shaped such that when the user's lips and/or teeth are transversely engaged with the intermediate portion, the mist-generating location is at least 20% deep, or at least 30% deep, or at least 40% deep, or at least 50% deep, or at least 60% deep, or at least 70% deep, or at least 80% deep, into an oral-cavity volume beneath the user's hard palate.

In some embodiments, a kit can comprise the inhalation device according to any of the embodiments disclosed hereinabove, packaged in a container such that the proximal portion that includes a liquid inlet is detached from the neck portion.

According to embodiments disclosed herein, an electrically-powered inhalation device for delivery of an aerosol to the oropharynx of a user comprises: (a) a distal portion including (i) an aerosol outlet defining a mist-exiting location and (ii) a piezo assembly including an ultrasonically vibrable mesh membrane, for producing, upon electrical activation, a mist comprising droplets of the liquid, the mesh membrane defining a mist-generating location; and (b) a neck portion including a narrow section, the narrow section being characterized by a minimum cross-sectional dimension that is at least 10% smaller than a minimum cross-sectional dimension passing through and parallel to the mesh membrane, a center of gravity of the inhalation device being displaced proximally from a distal end of the narrow section when the inhalation device is in a liquid-empty state, wherein the inhalation device is shaped such that when the user's lips and/or teeth are transversely engaged with the narrow section, the mist-generating location resides within the user's oral cavity and the mist-exiting location is in direct fluid communication with the user's oropharynx.

In some embodiments, the distal portion can comprise a distal casing encompassing the mesh membrane at least circumferentially.

In some embodiments, the narrow section can be characterized by a minimum cross-sectional dimension that is at least 20% smaller than the minimum cross-sectional dimension of the distal portion passing through and parallel to the mesh membrane, or at least 30% smaller than the minimum cross-sectional dimension of the distal portion passing through and parallel to the mesh membrane, or at least 40% smaller than the minimum cross-sectional dimension of the distal portion passing through and parallel to the mesh membrane, or at least 50% smaller than the minimum cross-sectional dimension of the distal portion passing through and parallel to the mesh membrane.

In some embodiments, the minimum cross-sectional dimension of the narrow section and the minimum cross-sectional dimension of the distal portion can define vectors that are coplanar, or within ±15° of being coplanar, or within ±30° of being coplanar, or within ±45° of being coplanar.

In some embodiments, the inhalation device can comprise a proximal portion that includes a power source for powering the piezo assembly.

In some embodiments, the inhalation device can comprise a proximal portion that includes a liquid inlet.

In some embodiments, the inhalation device can comprise a first proximal portion that includes a liquid inlet and a second proximal portion that includes a power source for powering the piezo assembly. In some embodiments, it can be that an outlet of the proximal portion that includes a liquid inlet is detachably attachable to the neck portion such that an interior volume of the proximal portion that includes a liquid inlet is arranged to be in fluid communication with an interior volume of the neck portion when a pressure-activated one-way valve is activated by pressure from the proximal portion that includes a liquid inlet.

In some embodiments, at least a part of the narrow section can be displaced proximally from the mesh membrane by at least 0.5 cm and not more than 6 cm, or by at least 0.5 cm and not more than 5.5 cm, or by at least 0.5 cm and not more than 5 cm, or by at least 0.5 cm and not more than 4.5 cm, or by at least 0.5 cm and not more than 4 cm, or by at least 1 cm and not more than 6 cm, or by at least 1 cm and not more than 5.5 cm, or by at least 1 cm and not more than 5 cm, or by at least 1 cm and not more than 4.5 cm, or by at least 1 cm and not more than 4 cm.

In some embodiments, the inhalation device can additionally comprise an inhalation sensor for monitoring a flow in an inhalation flow-path. In some such embodiments, the inhalation sensor can be effective to detect an air pressure in the inhalation-flow path.

In some embodiments, the inhalation sensor can be effective to detect a difference between an air pressure in the inhalation flow-path and an ambient air pressure outside the inhalation device.

In some embodiments, the inhalation device can comprise control circuitry configured to initiate and/or cease activation of the mesh membrane in response to a result of the monitoring of the flow in the inhalation flow path.

In some embodiments, the distal portion can comprise a liquid-retaining compartment in fluid communication with the neck portion, the liquid-retaining compartment being shaped to receive a quantity of the liquid from the neck portion by force of gravity when the inhalation device is in a first orientation, and to retain at least a part of the quantity against the force of gravity when the inhalation device is in a second orientation. In some such embodiments, the retaining can be by a wall of the liquid-retaining compartment, the wall being effective to partially block an egress of the retained at least a part of the quantity. In some embodiments, the second orientation can be such that substantially all of the mesh membrane is in liquid communication with the retained at least a part of the quantity. In some embodiments, the second orientation can be such that a surface liquid level in the liquid-retaining compartment is higher than a surface liquid level in the container.

In some embodiments, the inhalation device can be shaped such that when the user's lips and/or teeth are transversely engaged with the intermediate portion, the mist-generating location is at least 20% deep or at least 30% deep or at least 40% deep or at least 50% deep or at least 60% deep or at least 70% deep or at least 80% deep into an oral-cavity volume beneath the user's hard palate.

In some embodiments, a kit can comprise the inhalation device according to any of the embodiments disclosed hereinabove, packaged in a container such that the proximal portion that includes a liquid inlet is detached from the neck portion.

### BRIEF DESCRIPTION OF THE DRAWINGS

FIG. 1A is a schematic elevation drawing of an inhalation device, according to embodiments of the present invention.

FIG. 1B shows the inhalation device of FIG. 1A together with optional wick and removable liquid container, according to embodiments of the present invention.

FIG. 1C shows the inhalation device of FIG. 1B, with optional removal liquid container mated thereto, according to embodiments of the present invention.

FIG. 2A shows the inhalation device of FIG. 1C, in situ, in an activated state producing a mist in a user's oral cavity, according to embodiments of the present invention.

FIG. 2B schematically illustrates percentages of deepness into the volume beneath the hard palate.

FIG. 3 is a schematic elevation drawing of an inhalation device having a compact design, according to embodiments of the present invention.

FIG. 4 shows the inhalation device of FIG. 3, in situ, in an activated state producing a mist in a user's oral cavity, according to embodiments of the present invention.

FIG. 5 shows an inhalation device having inhalation and exhalation conveyances, in situ, in an activated state producing a mist in a user's oral cavity, according to embodiments of the present invention.

FIGS. 6A-6C show schematic views of an inhalation device according to embodiments of the present invention.

FIGS. 7A-7B show schematic views of an inhalation device according to embodiments of the present invention.

FIG. 8 shows an inhalation device according to embodiments of the present invention.

FIGS. 9A-D are schematic cross-sectional illustrations of the inhalation device of FIG. 8 and a liquid, according to embodiments of the present invention.

FIGS. 10A and 10B are cross-sectional views of inhalation devices according to embodiments of the present invention, showing liquid conduits having, respectively, circular and oval cross-sections.

FIG. 10C is a partial cutaway view of the proximal end of an inhalation device according to embodiments of the present invention, showing inhalation and exhalation sensors.

FIGS. 11A and 11B are schematic cross-sectional illustrations of an inhalation device having a distal liquid-storage volume, according to embodiments of the present invention, at two respective orientations.

FIGS. 12A and 12B are, respectively, schematic top- and side-view illustrations of an inhalation device having a display screen affixed to an intermediate portion of the inhalation device, according to embodiments of the present invention.

FIGS. 13A and 13B are, respectively, schematic top- and side-view illustrations of an inhalation device having a display screen affixed to a proximal portion of the inhalation device, according to embodiments of the present invention.

FIG. 14 is an annotated schematic cross-sectional illustration of the inhalation device of FIGS. 9A-D.

FIGS. 15A and 15B show schematic views of an inhalation device according to embodiments of the present invention, respectively assembled and unassembled, according to embodiments of the present invention.

FIG. 16 is an annotated schematic cross-sectional illustration of the inhalation device of FIGS. 15A-15B.

FIGS. 17A and 17B are schematic illustrations of kits including inhalation devices, according to embodiments of the present invention.

### DETAILED DESCRIPTION OF THE ILLUSTRATED EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

Following is a list of reference numbers used in the figures for physiological features:
10—oral cavity
15—lips
20—teeth
25—tongue
30—hard palate
40—nasal cavity
50—oropharynx
60—velo-pharyngeal port
70—pharyngeal cavity

Note: Throughout this disclosure, subscripted reference numbers (e.g., 10₁ or 10_{A}) may be used to designate multiple separate appearances of elements of a single species, whether in a drawing or not: for example: 10₁ is a single appearance (out of a plurality of appearances) of element 10. The same elements can alternatively be referred to without subscript (e.g., 10 and not 10₁) when not referring to a specific one of the multiple separate appearances, i.e., to the species in general.

For convenience, in the context of the description herein, various terms are presented here. To the extent that definitions are provided, explicitly or implicitly, here or elsewhere in this application, such definitions are understood to be consistent with the usage of the defined terms by those of skill in the pertinent art(s). Furthermore, such definitions are to be construed in the broadest possible sense consistent with such usage. Physiological terms as used herein are to be understood according to their generally accepted meanings.

The terms ‘aerosol’ and ‘mist’ as used herein are synonymous and are used to describe a suspension of liquid droplets in air. The terms ‘inhalation device’ and ‘inhaler’ as used herein are synonymous and are used to describe a device that delivers an aerosol to a user's oral cavity.

An inhalation device is disclosed herein for delivering an aerosol of a liquid well inside the user's oral cavity such that the device largely prevents the user's tongue from interfering with the delivery of the aerosol to the user's oropharynx. The exemplary devices disclosed herein use a piezo assembly that includes an ultrasonically vibrable mesh membrane to generate the aerosol, and so the piezo assembly has an aerosol outlet that in intended use will release the aerosol where desired.

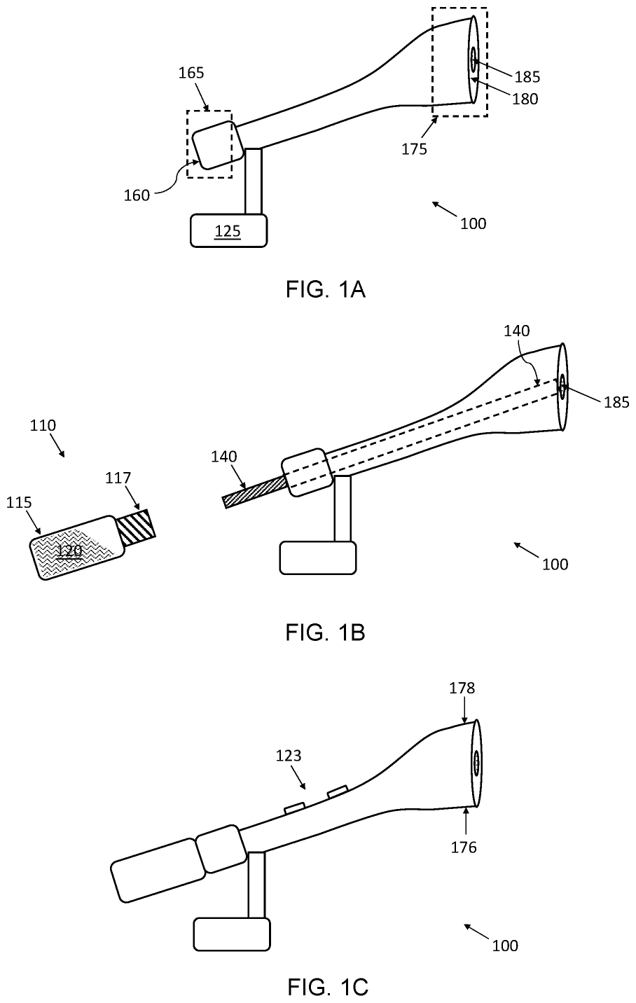

Referring now to the figures and in particular to FIGS. 1A, 1B and 1C, an inhalation device 100 according to embodiments is illustrated schematically. As seen in FIG. 1A, an inhalation device 100 has a distal portion 175 which includes the distal end of the inhalation device 100. The term ‘distal end’ is used herein to mean the end of the inhaler 100 at which an aerosol exits the inhaler 100. During normal intended use, the distal end is farthest from a user's hand, and/or is the first part of the device that enters a user's oral cavity. The term ‘distal’ may also used herein to indicate a direction towards the distal end. ‘Proximal’ as used herein refers to the end or direction which is opposite to the distal end or direction. The device 100 also includes a proximal portion 165. It should be noted that in some contexts the terms proximal portion and distal portion may be understood more broadly than the specific respective portions demarcated in FIG. 1A, and can refer to any portion that includes the respective end of the device.

FIGS. 1A-C show side elevation views, such that according to embodiments, the ‘top’ of the device 100 in each of the figures is the intended ‘top’ of the device 100 in actual use. Thus, in embodiments, an upper surface 178 of the distal portion 175 is intended to be ‘on top’ during use, and the lower surface 176 is intended to be ‘on the bottom’ during use. Nonetheless, in some embodiments, the inhalation device 100 is usable in other positions, e.g., with top and bottom reversed. The shape of the device 100 throughout the figures is shown as asymmetrical, i.e., the top of the device has a different contour than the bottom of the device. This can be beneficial for conforming to a user's oral cavity. Nonetheless, in some embodiments, the shape is symmetrical and does not have different contours on the top and the bottom of the device 100. In addition, the ‘thickness’ (dimension from top to bottom) of the distal portion 175 is shown as substantially thicker than the thickness of some of, most of, or all of the proximal portion 165. In embodiments, the distal portion 175 can have a thickness (e.g., maximum thickness, minimum thickness or average thickness) at least 30% or at least 50% or at least 100% greater than a corresponding thickness (respectively, maximum thickness, minimum thickness or average thickness) of the proximal portion 165. The relative thickness of the distal portion 175 can be useful in encouraging the user to place the distal portion 175 on top of the tongue so as to allow a mist generated by the device to directly reach the oropharynx.

The distal portion 175 includes a piezo assembly 180 that includes an ultrasonically vibrable mesh membrane 185. The mesh membrane 185 is the location at which an aerosol is generated/produced. In some embodiments (not shown) the distal portion can include an aerosol outlet displaced distally from the mesh membrane 185, where the the aerosol exits the device 100 via such an aerosol outlet. This can be useful, for example for bringing the aerosol closer to the user's oropharynx or for directing the generated mist in a specific direction. Thus, in some embodiments the ‘mist-generating’ location and the ‘mist-exiting’ location are the same location (for example, in FIGS. 1A-1C) and in some embodiments these two locations are displaced from each other.

In embodiments, the proximal portion 165 can include a liquid inlet 160, through which a liquid 120 can be introduced into the device 100 for producing the mist. The liquid inlet is preferably mateable with a source of the liquid 120. In the example shown in FIG. 1B, the source of liquid is a replaceable/removable (i.e., attachable/detachable) container 110. The term ‘mateable’ is used herein to indicate that a mating arrangement exists, e.g., corresponding threading, snap-closures or appropriately sized inlet-outlet diameters. According to the example, the container 110 has a liquid storage volume 115 and an outlet 117. The outlet 117 is mateable with the liquid-inlet 160 of the device 100. FIG. 1B also shows a capillary pathway 140 for distally transporting liquid 120 in the direction of the mesh membrane 185. The capillary pathway 140 is typically disposed, and optionally held, so that a distal portion thereof is in contact with the mesh membrane 185, or displaced no more than 2 mm or no more than 1 mm from the mesh membrane 185. A proximal portion of the capillary pathway 140 is generally disposed within the liquid-storage volume 115 of the container 110 so as to establish a pathway for water transport from the liquid-storage volume 115 to the mesh membrane 85. In some embodiments, the container 110 is formed and/or provided as a component of the inhalation device 100.

A ‘capillary pathway’ 140 as the term is used herein is a material suitable for transport of a liquid) along a pathway by capillary action. Such a material often includes fibers, such as plant-based fibers e.g., cellulose, polymer-based fibers e.g., polyester, glass fibers e.g., in a woven fabric or bundled or unbundled glass fibers, or carbon fibers. In some non-limiting examples, the fibers can be very small, i.e., having diameters in the range of several or tens of microns. In other examples, the fibers can be larger. While the term “pathway” may appear to imply that a pathway for liquid transport to a mesh membrane may be a direct path, that is not necessarily the case. The transport of a liquid through the capillary pathway may include progression in random directions or omnidirectional progression. In some embodiments, the capillary pathway 140 can include fibers arranged so as to form direct pathways from various parts of the liquid-storage volume 115 but this is not necessary for the capillary transport to be effective. In some embodiments, the capillary pathway can comprise a hydrophilic material that is effective to facilitate transport of an aqueous liquid.

As shown in FIG. 1A, an inhalation device 100 according to embodiments can include comfort element(s) 123 for ease of placement of teeth and/or lips. The two bumps shown in FIG. 1C are just one non-limiting example of such comfort elements; other non-limiting examples include depressions and single bumps. Such elements are not present in all designs within the scope of the present invention, but can be useful in some cases for optimal positioning of the device 100, and especially positioning of the distal portion 175, within the oral cavity. As shown in FIG. 1A. an inhalation device 100 according to embodiments can include a power and electronics module 125, which can include, for example, a power source (e.g., a battery or connection for mains electricity), wireless communication arrangements, and/or control circuitry. The control circuitry can include electronic hardware such as a printed circuit board, and firmware or software for operation of the device.

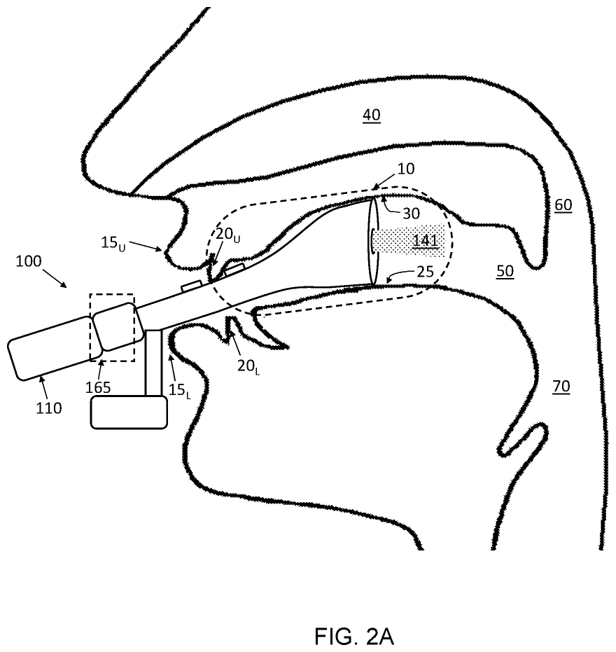

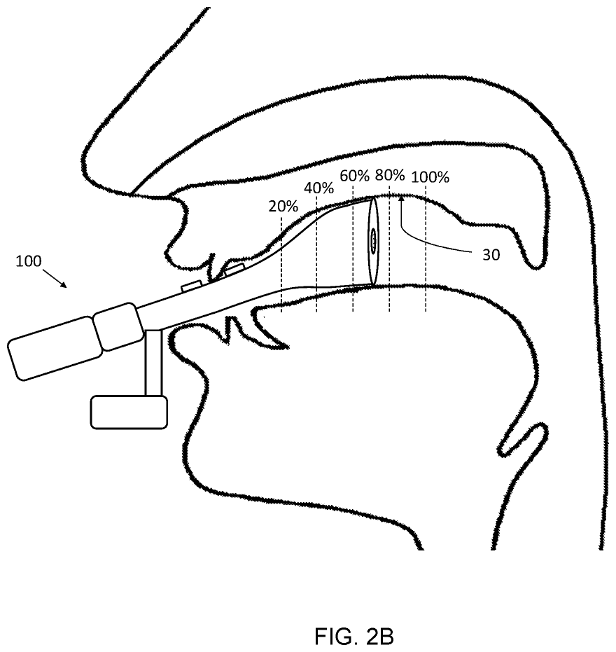

Referring now to FIG. 2A, the in-situ placement of an inhalation device 100 according to embodiments (and according to the example of FIGS. 1A-C) is shown with respect to a user's oral cavity 10 and mouth parts such as upper and lower teeth 20_{U}, 20_{L}, upper and lower lips 15_{U}, 15_{L}, tongue 25 and hard palate 30. The inhalation device 150 is preferably dimensioned such that the distal portion 175 spans the oral cavity 10 from the tongue 25 to the hard palate 30. Teeth 20 and/or lips 15 can close on the device 100 at teeth-engaging portions or lip-engaging portions that are distally displaced from the proximal portion 165, and thus help to maintain the position of the device 100 as illustrated. The comfort elements 123 illustrated one non-limiting example of a teeth-engaging portion. Mist 141, as shown schematically in FIG. 2A, is produced at the mesh membrane 185: as mentioned hereinabove, the mist-generating location and mist-exiting location (i.e., the aerosol exit from inhaler 100) are the same location in this exemplary design. In other words, not only is the aerosol outlet located in the distal portion 175 of the inhalation device 100, in this design the mesh membrane 185 is also located in the distal portion 175. Thus when positioned as illustrated with the top surface of 178 of the distal portion 175 in contact with the hard palate 30, and the bottom surface of 176 of the distal portion 175 in contact with the tongue 20, both the mesh membrane and the aerosol exit are placed in fluid communication with the user's oropharynx 50. A proximal air inlet (not shown in FIG. 2A) can be added for ensuring that proper inhalation can still occur when lips 15 are closed around the device 100.

In embodiments, the distal portion 175 of the inhalation device 150 can comprise a coating for generating a taste and/or odor sensation for the user. The coating can be applied, for example, on the tongue-contacting portion of the distal portion.

In another design example, which is not illustrated, the inhaler 100 of FIGS. 1-2 can be formed to be shorter, so that the container 110 is part of the proximal portion and the teeth-engaging and/or lip-engaging location is on a surface of the container 110.

In embodiments, the distal portion 175 of the inhalation device 150 includes the mist-generating location, i.e., the mesh membrane 185, and the device 150 is formed so that the mist-generating location is beneath the hard palate 30. In some embodiments, the volume of the oral cavity beneath the hard palate can be demarcated according to ‘hard-palate-deepness’ as illustrated schematically in FIG. 2B. For example, the mist-generating location can be in the deeper half of the volume beneath the hard palate 30—or in the deepest 40% in the example of FIG. 2B, or in the deepest 20% (not shown). In other words, the mist-generating location (and the mist-exiting location) can be at least 50% deep into the volume beneath the hard palate 30 or at least 60% deep or at least 70% deep or at least 80% deep. In other embodiments, the mist-generating location might not be quite as deep—for example, the mist-generating location can be at least 20% deep or at least 30% deep or at least 40% deep into the volume beneath the hard palate. Greater ‘deepness’ can be advantageous so as to shorten the path of fluid communication between the mist-exiting location and the oropharynx.

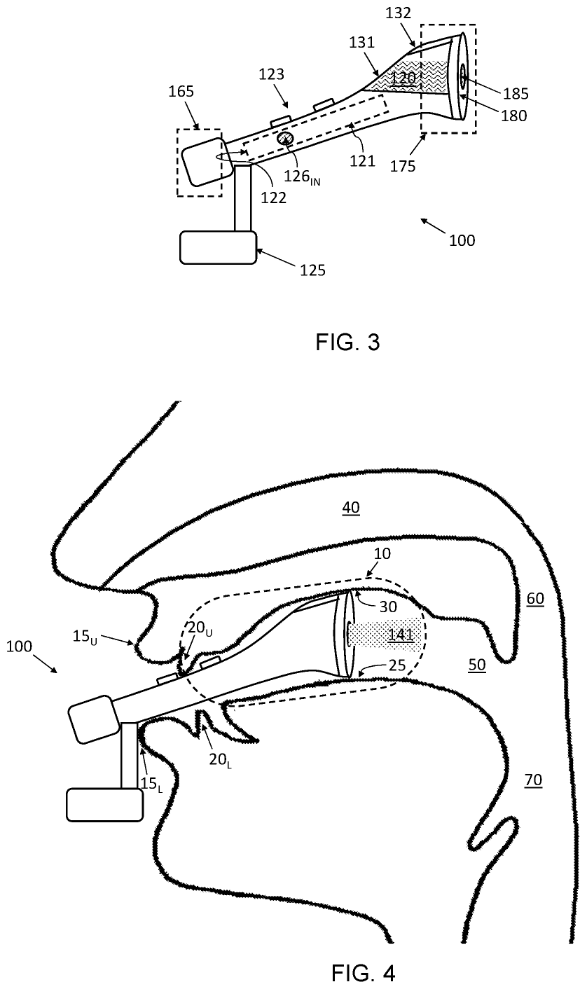

We now refer to FIGS. 3 and 4.

FIG. 3 illustrates a more compact design for an inhalation device 100 according to embodiments of the present invention, and FIG. 4 shows the in-situ placement of the device 100. Like the inhaler 100 of FIGS. 1A-2, the inhaler 100 of FIGS. 3 and 4 has a distal portion 175 (comprising the piezo assembly 180 and the aerosol outlet which happens to be co-located with mesh membrane 185) and a proximal portion 165, power and electronics module 125, and optional comfort elements 123. Liquid 120 for producing therefrom a mist is stored in compartment 131 (which is optionally detachable/attachable). Compartment 131 has a opening for filling and refilling: the compartment 131 has an openable closing element 132. The inhalation device 100 of FIG. 3 includes an airflow channel 121 having a proximal air inlet 122 for ensuring that proper inhalation can still occur when lips 15 are closed around the device 100. As can be seen in FIG. 4, the air inlet 122 is positioned so as to remain outside the lips 15 when the device 100 is positioned for operation in situ. Referring again to FIG. 3, an ‘inhalation sensor’, i.e., flowmeter or airflow sensor 126_{IN} is provided for activating the piezo assembly 180 upon detection of inhalation. In embodiments, a piezo assembly 180 can be activated to produce a mist (in the presence of liquid) manually, e.g., by control circuitry in response to a user pressing a button or moving a switch, and/or automatically by control circuitry (e.g., in power and electronics module 125) monitoring the inhalation sensor 126_{IN} for indication of an inhalation airflow. In some embodiments, the inhalation sensor 126_{IN} is configured to detect an air pressure. In some embodiments, the inhalation sensor 126_{IN} is configured to detect a difference between an air pressure in the inhalation flow-path and an ambient air pressure outside the inhalation device

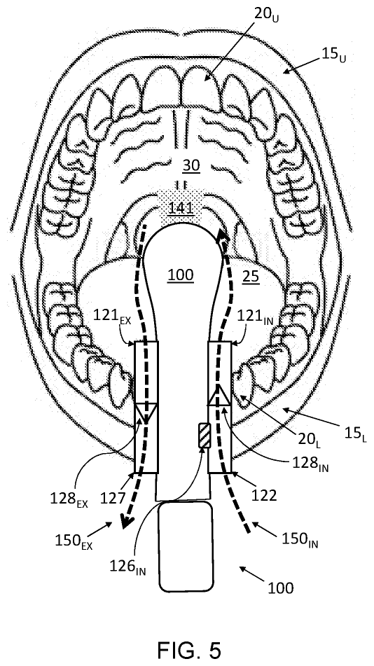

Referring now to FIG. 5, an inhalation device 100 is shown in-situ from a different angle than that of FIGS. 2A-B and 4. Two airflow channels 121, 129 are provided for conveyance of an inhalation airflow (indicated by arrow 150_{IN}) and an exhalation airflow (indicated by arrow 150_{EX}), respectively. As also shown in FIG. 3, the inhalation airflow-channel 121 of FIG. 5 includes an air-inlet 122 positioned beyond user's lips 15 outside of a potentially closed mouth. Similarly, the exhalation airflow-channel 129 includes an exhaust outlet 127 positioned beyond the user's lips 15. In embodiments, each of the airflow channels 121_{IN}, 121_{EX} can be equipped with respective one-way fluid valves 128_{IN}, 128_{EX} which by their presence define the directionality of airflow within each respective airflow-channel. An inhalation sensor 126_{IN}, e.g., a flowmeter or air-pressure sensor, can be provided for monitoring and detecting the presence of an inhalation breath, so that control circuitry can activate or deactivate or otherwise modify the mist-generation of the mesh membrane 180. In embodiments, the mesh membrane can be effective to eject at least 5 times, or at least 10 times, or at least 20 times, or at least 50 times more liquid 120 in the mist 141 during user inhalation than during user exhalation. It will be apparent to those skilled in the art that it does not matter which of the airflow channels 121 is used for inhalation and which is used for exhalation, and the labeling in the figures is merely for convenience.

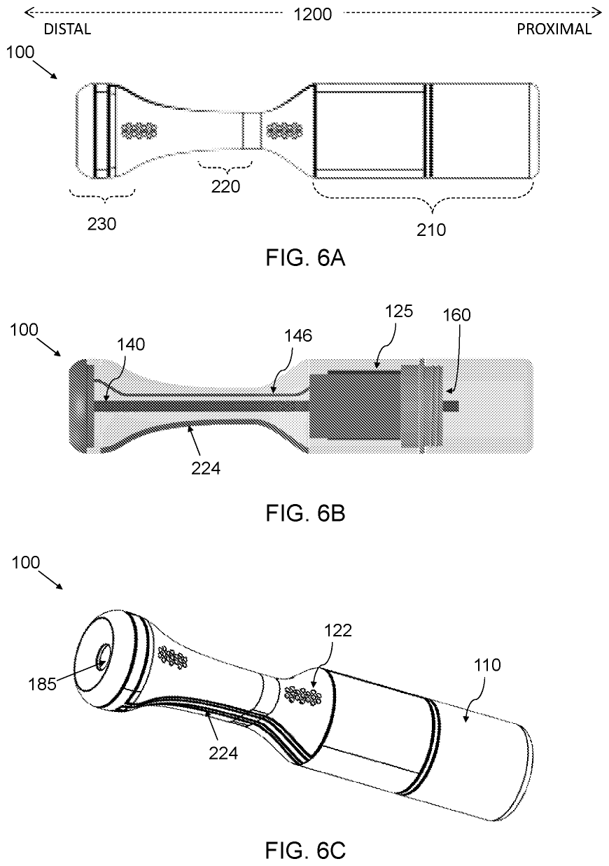

We now refer to FIGS. 6A to 6C, which show various schematic views of an inhalation device 100 according to embodiments, including embodiments already described hereinabove. Respective distal and proximal directions are illustrated by arrow 1200.

As seen in the elevation view of FIG. 6A, the inhalation device 100 includes a distal portion 230, a proximal portion 210 and an intermediate portion 220 that is displaced proximally from the distal portion 230 and distally from proximal portion 210. The proportions of the respective portions 210, 220, 230 are entirely for illustration purposes only, and any of the respective portions 210, 220, 230 can be larger or smaller. In some examples, they can also be contiguous, i.e., without gaps between the various portions. When the inhalation device 100 is in use according to preferred use modes, the intermediate portion 220 is contacted, i.e., transversely engaged, by a user's lips 15 and or teeth 20, and the distal portion 230, which includes the mist-generating mesh 185, is disposed within the user's oral cavity 10.

FIG. 6B, where the outer envelope of the inhalation device 100 is made ‘transparent’, schematically illustrates typical internal components of the inhalation device of FIG. 6A: capillary pathway 140 leading from liquid inlet 160 (where a container or compartment, not shown, would hold a quantity of a liquid) to the mesh 185, and electrical wire(s) 146 leading from control circuitry and power supply 125 to the mesh 185.

As shown in FIG. 6C, the inhalation device 100 can include air-inlet holes 122 which are proximal of the intermediate portion 220 such that the air-inlet holes 122 are outside the mouth. A taste-producing surface section 224 can be provided in the distal portion 230 and/or the intermediate portion 220. The taste-producing surface section 224 is preferably on the ‘bottom’ of the inhalation device 100 during use so as to bring the taste-producing surface section 224 into contact with the user's tongue 25.

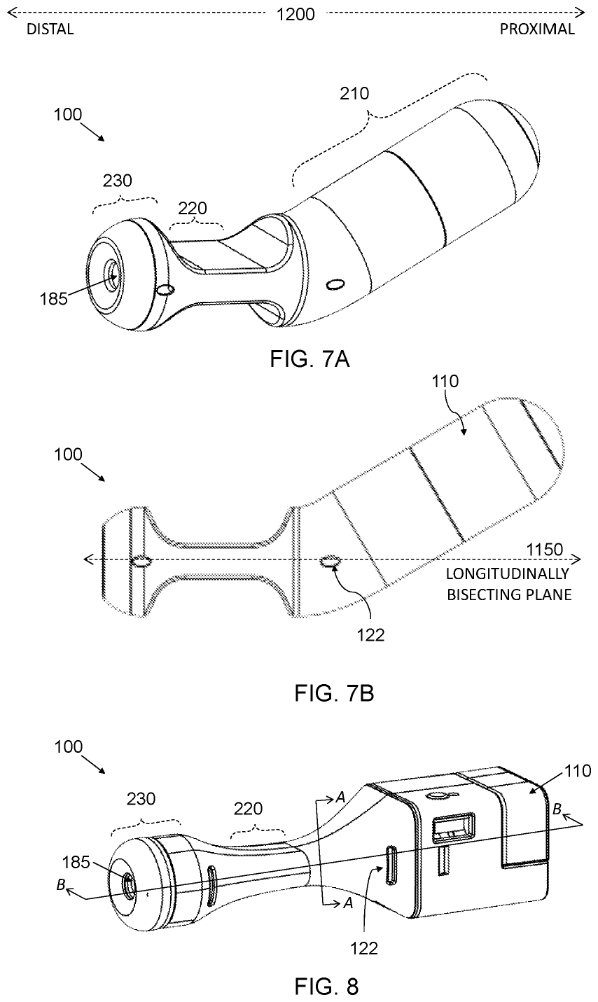

Referring now to FIGS. 7A and 7B, another inhalation device 100 according to embodiments is illustrated. The inhalation device of FIGS. 7A-B can operate effectively without a capillary pathway for transport of liquid from the container 110 to the mesh 185 because of a gravity-aided design. A plane 1150 is shown longitudinally bisecting the intermediate portion 220 (and/or the distal portion 230). When the horizontally-bisecting plane 1150 is held horizontal, e.g., parallel to a floor (not shown), the container 110 is held higher than the plane 1150 and therefore higher than the mesh 185, so that liquid can be made by gravity to flow to the mesh 185. While FIG. 7B shows the entire container 110 as being higher than the plane, in some designs it can be that a portion of the container higher than the plane.

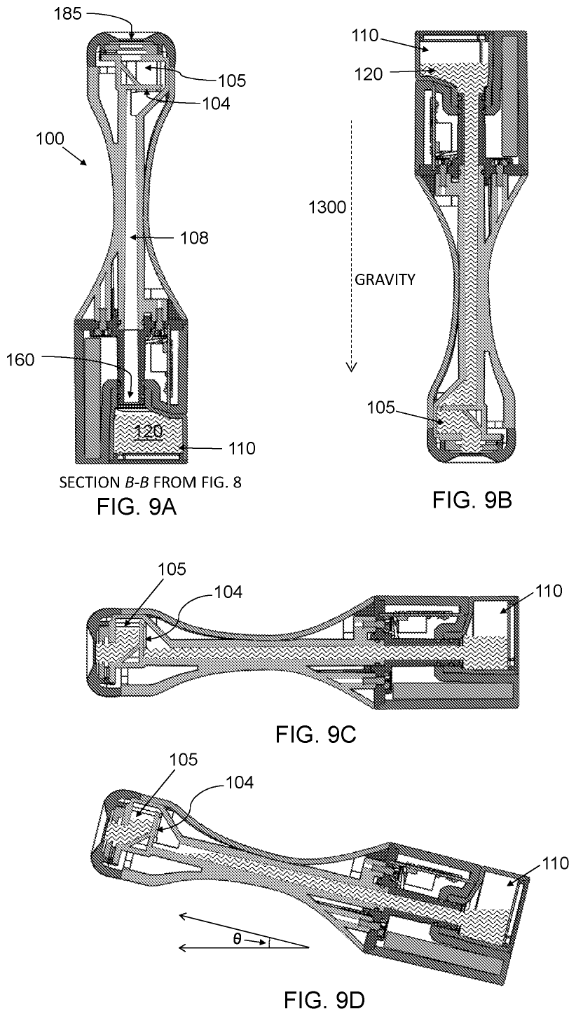

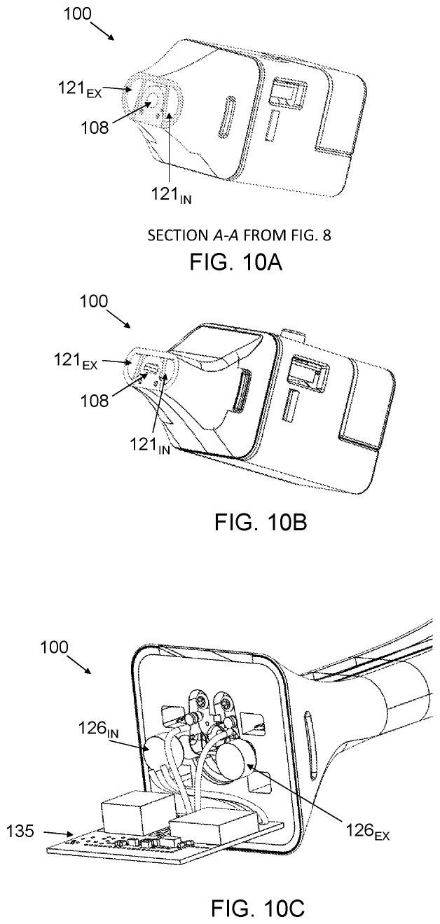

FIG. 8 illustrates another inhalation device 100 according to embodiments, wherein the container 110 does not extend across the entire proximal portion 210 of the inhalation device 100. Features of the inhalation device 100 of FIG. 8 according to embodiments, are illustrated in the cross-sectional views of FIGS. 9A-D, which correspond to section B-B in FIG. 8. In FIG. 9A, a quantity of liquid 120 is disposed in a container 110 which is engaged with liquid-inlet 160. The distal portion 230 includes a liquid-retaining compartment 105 in fluid communication with the proximal liquid inlet 160 vis a liquid conduit 108, illustrated in FIGS. 9A-D as a connecting tube or pipe. The liquid-retaining compartment 105 is partially bounded on one side by a liquid-retaining wall 104. When the inhalation device 100 is turned upside-down as shown in FIG. 9B, the liquid 120 flows down with gravity (indicated by arrow: 1300) to fill the liquid-retaining compartment 105, as well as at least a portion of the liquid conduit 108. While FIG. 9B shows the entire liquid conduit 108 full of liquid, and a portion of the liquid remaining in the container 110, in other examples there can be more or less liquid 120 provided, and/or the relative capacities of the retaining compartment 105, the liquid conduit 108 and/or the container 110 can be larger or smaller than illustrated in FIG. 9B such that the liquid 120 fills the liquid-retaining compartment 105 but only some or none of the liquid conduit 108, such that the container 110 is emptied in such examples.

FIG. 9C illustrates the function of the liquid-retaining wall 104 that partially bounds the liquid-retaining compartment 105. After the reversing of the inhalation device as shown in FIG. 9B, the inhalation can be brought to a horizontal position for use as shown in FIG. 9C, which causes the liquid in the connected reservoirs of the liquid-retaining compartment 105 and the container 110 to tend to ‘seek its own level’. However, the liquid-retaining wall 104 prevents a portion of the liquid 120 delivered to the liquid-retaining compartment 105 (during the reversing of the inhalation device 100) from leaving the liquid-retaining compartment 105 after the inhalation device 100 is turned horizontal, or, as illustrated in FIG. 9D, ‘below horizontal’. The liquid 120 in the liquid-retaining compartment 105 can thus be ‘cut off’ from the remainder of the liquid in the container 110 and conduit 108. The height of the liquid-retaining wall 104 is preferably sufficient to ensure that for a range of angles 0 (horizontal, e.g., as in FIG. 9C) to θ) (e.g., as in FIG. 9D), the mesh 185 is kept in contact with liquid 120 retained in the liquid-retaining compartment 105. Setting the value of θ is a design choice which reflects a desired range of angles at which the inhalation device 100 can work effectively. The height of the liquid-retaining wall 104 should be sufficient to retain liquid 120 in the compartment 105 through the range of angles 0 to θ even though the surface level of the liquid 120 in the compartment 105 is higher than in the container 110, as illustrated in the example of FIG. 9D. During normal operation, whenever enough liquid 120 is nebulized out of the inhalation device to cause a portion or substantial portion, e.g., over 1 mm, of the mesh 185 to no longer be in contact with liquid 120, the user can simply upend the inhalation device (as in FIG. 9B) to ‘refill’ the liquid-retaining compartment 105 from the remaining liquid 120 in the container 110 and conduit 108. and then restore the comfortable use position of FIG. 9C or FIG. 9D.

In embodiments, a maximum retainable fluid capacity of the liquid-retaining compartment 105 (i.e., the quantity of the liquid 120 retained by the liquid-retaining wall 104) is at least 0.5 cc and not more than 4 cc. In some embodiments, the maximum retainable fluid capacity of the liquid-retaining compartment 105 is at least 1 cc and not more 3 cc. In some embodiments, the maximum retainable fluid capacity of the liquid-retaining compartment 105 is at least 1.5 cc and not more 2.5 cc. A ratio of (i) a combined fluid capacity of the container 110 and the conduit 108 to (ii) the maximum retainable fluid capacity of the liquid-retaining compartment 105, is at least 1 and not more than 4. In some embodiments, this ratio is at least 1.5 and not more than 3. In some embodiments, this ratio is at least 1.75 and not more than 2.5.

Reference is made to FIGS. 10A, 10B and 10C, both of which show cross-sectional views corresponding to section A-A in FIG. 8 such that the liquid conduit 108 and respective airflow channels 121_{IN}, 121_{EX} can be seen. The liquid conduit 108 of FIG. 10A has a circular cross-section. The liquid conduit 108 of FIG. 10B has an oval cross-section, which, inter alia, can be effective to reduce turbulent flow within the liquid conduit 108.

FIG. 10C shows respective inhalation and exhalations sensors 126_{IN}, 126_{EX}. When present each of the sensors 126 is in communication with a respective flow path 121. In some embodiments, only one of the sensors 126 is present.

The inhalation sensor 126_{IN} is provided for monitoring a flow in an inhalation flow-path, e.g., inhalation flow path 121_{IN}. In an example, the inhalation sensor 126_{IN} can be effective to detect an air pressure in the inhalation-flow path 121_{IN}. In another example, the inhalation sensor can be effective to detect a difference between an air pressure in the inhalation flow-path 121_{IN} and an ambient air pressure outside the inhalation device 100. In some embodiments, the control circuitry 135 is configured to initiate and/or cease activation of the mesh membrane 185 in response to a result of the monitoring of the flow in the inhalation-path 121_{IN}.

The exhalation sensor 126_{EX} is for monitoring a flow in an exhalation-flow path, e.g., exhalation flow path 121_{EX}. In an example, the exhalation sensor 126_{EX} is configured to detect a concentration of a chemical compound in the exhalation-flow path 121_{EX}. In some embodiments, the chemical compound is a component of the liquid 120 which is misted by the inhalation device. In some embodiments, the chemical compound is a chemical compound of interest to a user. For example, a user may wish to know the concentration of an intoxicating chemical compound in an exhalation, such as, for example, and not exhaustively, alcohol or tetrahydrocannabinol. In another example, the chemical compound can be an indicator of a disease or of a current health condition of the user. In some embodiments, the control circuitry 135 is configured to cease or delay activation of the mesh membrane 185 in response to a result of the monitoring of the flow in the exhalation flow path 121_{EX}.

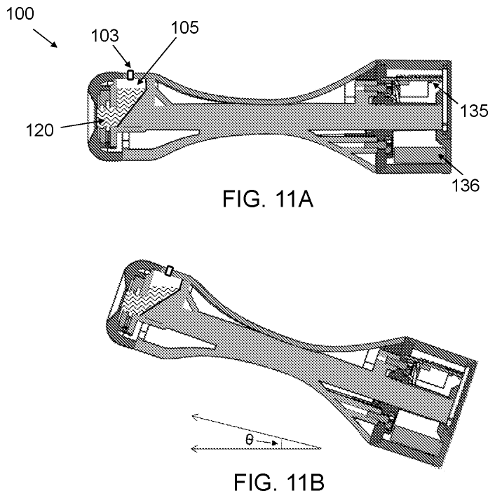

Referring now to FIGS. 11A and 11B: an inhalation device 100 comprises a distal liquid-storage compartment 105 fillable through filling port 103. In the design of FIGS. 11A and 11B, the inhalation device 100 does not include a proximal source of liquid 120, nor does it include a liquid conduit 108. Instead, the liquid in the distal liquid-storage compartment 105 is in contact with the mesh membrane. The distal liquid-storage compartment 105 is designed such that for a range of angles 0 (horizontal, e.g., as in FIG. 11A) to θ (e.g., as in FIG. 11B), the mesh 185 is kept in contact with liquid 120 retained in the liquid-storage compartment 105. Setting the value of θ is a design choice which reflects a desired range of angles at which the inhalation device 100 can work effectively.

It can be desirable to add a display screen (or, equivalently, any display device) to an inhalation device for visually communicating information to a user. The information to be communicated can include, for example, and not exhaustively: the quantity or percentage of liquid remaining; the quantity or percentage of a compound in the liquid that is remaining; the amount or percentage of liquid (or of the compound in the liquid) that has already been consumed by the delivery of the mist, with or without including prior any fills of the liquid; the identity of the compound: a power meter showing remaining battery life; a concentration of a compound detected in an exhalation airflow; whether a concentration of a substance in the exhalation airflow exceeds a preset limit for intoxication; and a health indicator such as the presence of a virus, bacteria, or any other health indicator that can be detected in an exhalation.

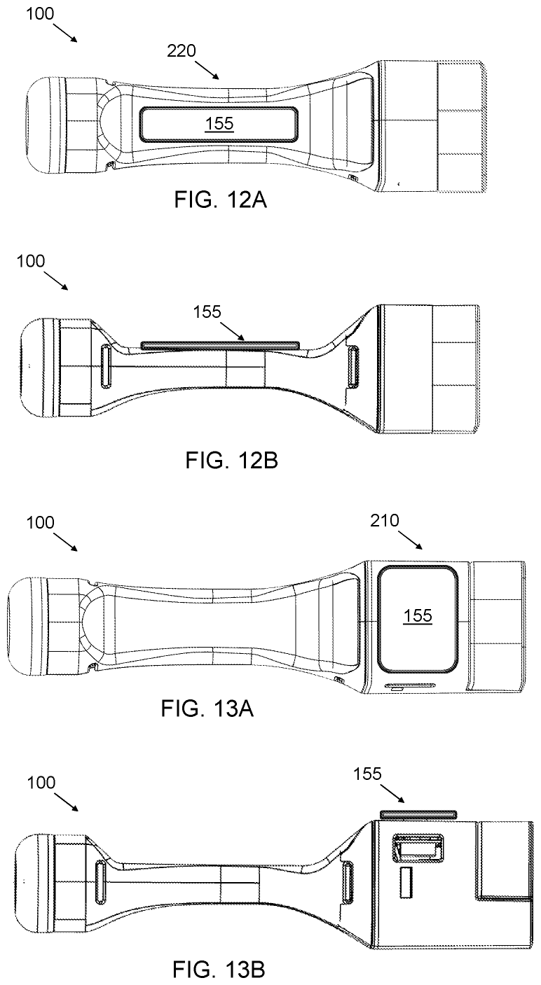

A display screen can be mounted to or installed on any convenient section of any of the inhalation devices 100 disclosed herein. FIGS. 12A and 12B (top and ide views, respectively of an inhalation device 100 according to embodiments) show a display screen 155 mounted to the intermediate portion 220 of the inhalation device 100. FIGS. 13A and 13B (top and ide views, respectively of an inhalation device 100 according to embodiments) show a display screen 155 mounted to the proximal portion 210 of the inhalation device 100.

In any of the embodiments disclosed herein, the liquid 120 can include a medicament. In some embodiments, the quantity of liquid 120 used to generate the mist 141 can be a based on a predetermined dosage. This can be accomplished by the control circuitry in accordance with previous programming or in response to a user input.

In any of the embodiments disclosed herein, a capillary pathway 140 may be used to transport liquid to the mesh membrane.

In any of the embodiments disclosed herein, the inhalation device 150 can be used ‘hands-free’, i.e., when the inhalation device 150 is disposed so that the user's teeth are engaged with a front-teeth-engaging portion distally displaced from the proximal portion 165, and/or the user's lips are engaged with a lip-engaging portion distally displaced from the proximal portion 165, the device 150 can be held in place by the user's lips 15 and or teeth 20 during activation/operation and mist-generation without having to use a hand to keep it in place.

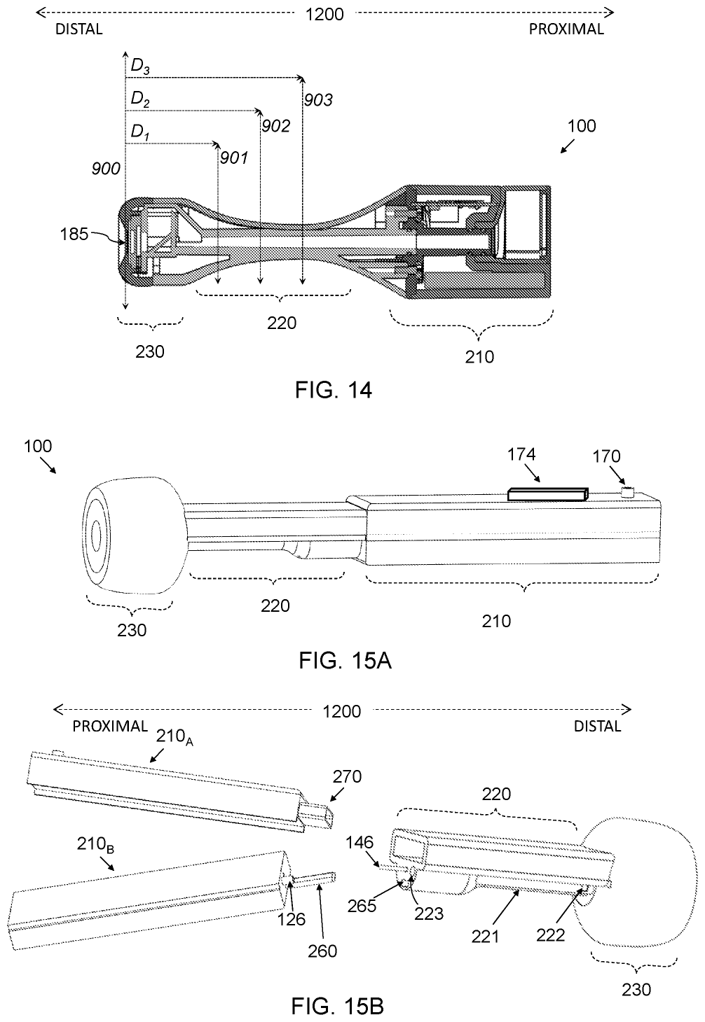

We now refer to FIG. 14. An inhalation device 100, e.g., the inhalation device 100 of FIGS. 9A-9D, is illustrated with annotations marking certain locations and dimensions according to embodiments. A plane indicated by arrow 900 passes through the mesh membrane 185 and is parallel thereto. A cross section of the distal portion 230 and through the mesh membrane 185 can be round, oval, or any other shape. (Distal and proximal directions are indicated by arrow 1200.) A minimum dimension of the cross-section, i.e., measured by an arc or line segment that passes through a center-point of the cross-section is a minimum cross-sectional dimension of the distal portion 230 passing through and parallel to the mesh membrane 185. The mesh membrane, in embodiments, is encompassed, at least circumferentially, by a distal casing 190. A neck portion 320 of the inhalation device 100, analogous to the intermediate portion 220 shown, e.g., in FIGS. 6A and 7A, is located proximal to the distal portion 230. In some designs, the neck portion 320 is contiguous with the distal portion 230 and in some designs there can be additional inhaler length in between that for purposes of this disclosure has the distinguishing features of neither the distal portion nor the neck portion, and can be arbitrarily assigned to either one.

The neck portion 320 includes at least one narrow section. A narrow section can comprise a single point or a longer length of the neck portion 320. A narrow section is characterized by having a cross-section with a minimum dimension that is smaller, by a given margin, than the minimum cross-sectional dimension of the distal portion 230 passing through and parallel to the mesh membrane 185. For example, a minimum cross-sectional dimension in a narrow section can be at least 10% smaller than the minimum cross-sectional dimension of the distal portion 230 passing through and parallel to the mesh membrane 185. In other examples, the minimum cross-sectional dimension in a narrow section can be at least 20% smaller, or at least 30% smaller, or at least 40% smaller, or at least 50% smaller, or even smaller. The minimum cross-sectional dimension of the narrow section and the minimum cross-sectional dimension of the distal portion 230 define vectors that are coplanar, or within ±15° of being coplanar, or within ±30° of being coplanar, or within ±45° of being coplanar.

Three examples of narrow sections are shown in FIG. 14 where planes indicated by the arrows 901, 902 and 903 pass through the neck portion 320. Additionally or alternatively, the entire neck portion 320 can be considered as comprising one narrow section from one end of the neck portion 320 to the other, because in FIG. 14 the entire neck portion 320 clearly can be seen to be characterized by a minimum cross-sectional dimension that is at least 10% smaller than the minimum cross-sectional dimension of the distal portion 230 passing through and parallel to the mesh membrane 185. In this case, the first, i.e., distalmost point of the ‘narrow section’ meeting the 10%-smaller limitation can be is of interest, although in some embodiments all points of the narrow sections of the neck portion 320 can be of interest. For example the first (distalmost) annotated point in the neck portion 320 is defined by a cross-section at plane 901. As shown in FIG. 14, the plane 901 is proximally displaced from the mesh membrane 185, i.e., from the plane 900 passing through and parallel to the mesh membrane 185, by a distance indicated by the arrow D₁. In embodiments, this distance D₁ is at least 0.5 cm and not more than 5.5 cm, or at least 0.5 cm and not more than 5 cm, or at least 0.5 cm and not more than 4.5 cm, or at least 0.5 cm and not more than 4 cm, or at least 1 cm and not more than 6 cm, or at least 1 cm and not more than 5.5 cm, or at least 1 cm and not more than 5 cm, or at least 1 cm and not more than 4.5 cm, or at least 1 cm and not more than 4 cm. Similarly, if the narrow section at plane 902 were to be the first (distalmost) point meeting the 10%-smaller limitation, then the corresponding distance from the mesh membrane 185 (and plane 900) D₂ would be, in such a design, at least 0.5 cm and not more than 5.5 cm, or at least 0.5 cm and not more than 5 cm, or at least 0.5 cm and not more than 4.5 cm, or at least 0.5 cm and not more than 4 cm, or at least 1 cm and not more than 6 cm, or at least 1 cm and not more than 5.5 cm, or at least 1 cm and not more than 5 cm, or at least 1 cm and not more than 4.5 cm, or at least 1 cm and not more than 4 cm. Similarly, if the narrow section at plane 903 were to be the first (distalmost) point meeting the 10%-smaller limitation, then the corresponding distance from the mesh membrane 185 (and plane 900) D₃ would be, in such a design, at least 0.5 cm and not more than 5.5 cm, or at least 0.5 cm and not more than 5 cm, or at least 0.5 cm and not more than 4.5 cm, or at least 0.5 cm and not more than 4 cm, or at least 1 cm and not more than 6 cm, or at least 1 cm and not more than 5.5 cm, or at least 1 cm and not more than 5 cm, or at least 1 cm and not more than 4.5 cm, or at least 1 cm and not more than 4 cm. In other words, the first (distalmost) point in the narrow section, i.e., the distal end of a narrow section, which meets the 10%-smaller limitation is at a distance from the mesh membrane 185 (and plane 900) that falls in one of the ranges above, i.e., at least 0.5 cm and not more than 5.5 cm, or at least 0.5 cm and not more than 5 cm, or at least 0.5 cm and not more than 4.5 cm, or at least 0.5 cm and not more than 4 cm, or at least 1 cm and not more than 6 cm, or at least 1 cm and not more than 5.5 cm, or at least 1 cm and not more than 5 cm, or at least 1 cm and not more than 4.5 cm, or at least 1 cm and not more than 4 cm. In the foregoing, the 10%-smaller limitation is used as a non-limiting example, and in some embodiments the minimum cross-sectional dimension in a narrow section can be at least 10% smaller than the minimum cross-sectional dimension of the distal portion 230 passing through and parallel to the mesh membrane 185.

In embodiments, the inhalation device 100 is designed so that a center of gravity of the inhalation device 100 is proximal to the first (distalmost) point in the narrow section i.e., the distal end of a narrow section, which meets the 10%-smaller limitation, when the inhalation device 100 contains no liquid.

Referring now to FIGS. 15A and 15B, an inhalation device according to embodiments is schematically illustrated, respectively assembled and unassembled.

As shown in FIG. 15B, this design can be supplied in a modular configuration, where two proximal portions 210_{A}, 210_{B} are detachably attachable to/from an inhalation-device section (shown in FIG. 17B as 450) comprising both the distal portion 230 of the inhalation device 100 and the neck portion 320 of the inhalation device 100. A first proximal portion 210_{A} includes an interior liquid-holding volume (not shown) and can be used as a replaceable container for the inhalation device 100. The first proximal portion 210_{A} includes, according to some embodiments at least one of a liquid inlet 170 and a pressurable surface 174, e.g., a flexible surface that can be manually depressed so as to cause a liquid to flow out of the first proximal portion 210_{A} and into the neck portion 320 of the inhalation-device section 450. In some embodiments, the outlet 270 of the first proximal portion 210_{A} includes a pressure-activated one-way valve such as, in a non-limiting example, a duckbill valve. In some embodiments, at least an upper surface of first proximal portion 210_{A} is flexible and a separate pressurable surface 174 is unnecessary, and in some embodiments, the entire first proximal portion 210_{A} is flexible and a separate pressurable surface 174 is unnecessary.

An electronic and/or electrical connection 146 inserts into a corresponding hole (not shown) in the second proximal portion 210_{B} for powering the piezo assembly 180 from a power source 125 located in the second proximal portion 210_{B}. In embodiments, an inhalation sensor 126 for indication of an inhalation airflow is provided on the second proximal portion 210_{B} and inserts into the end 223 of an airflow channel 221 having a distal air inlet 222 located proximal to the distal portion 230. Thus, when a user holds the inhalation device in his mouth, e.g., as illustrated in FIGS. 2A-2B, mutatis mutandis, and the user's lips 15 close around the neck portion 320 of the inhalation device 100, an inhalation will draw air through the distal air inlet 222 and through the airflow channel 221, causing the inhalation sensor 126 to register a pressure drop and initialize the activation of the piezo assembly 180 and generate a mist. Similarly, the sensor 126 can register the cessation of an inhalation and cease the operation of the piezo assembly and the generation of the mist.

In embodiments, one or more stabilization pins 260 may be provided for making and stabilizing the connection of the second proximal portion 210_{B} and the inhalation-device section 450. The one or more stabilization pins 260 are arranged for being inserted into corresponding hole(s) 265. Additionally or alternatively, other features can be added for making and stabilizing the connection of the second proximal portion 210_{B} and the inhalation-device section 450, as well as the connection of the first proximal portion 210_{A} and the inhalation-device section 450.

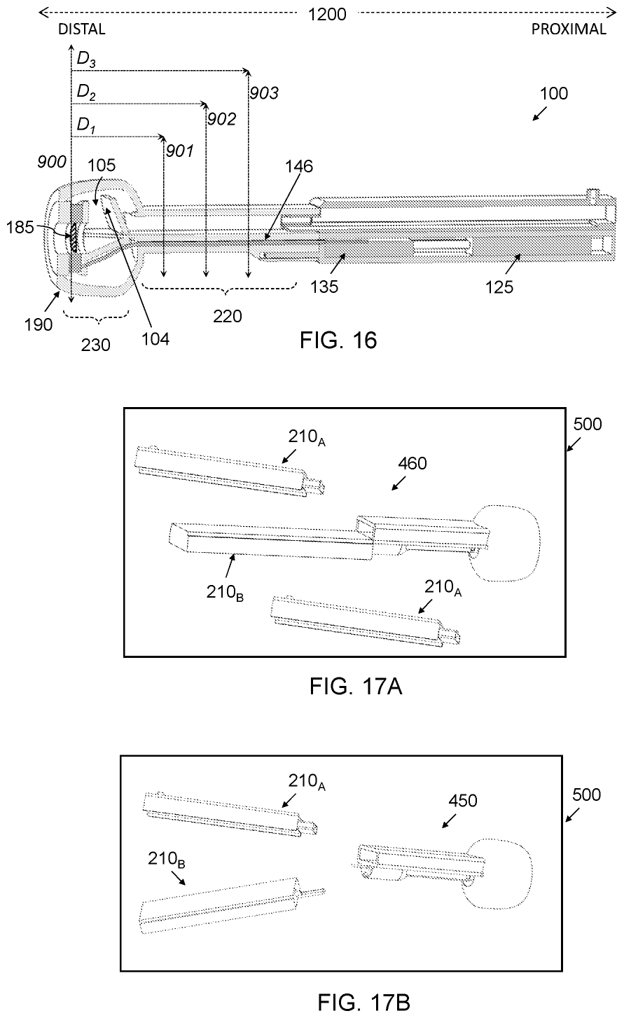

FIG. 16 shows a schematic cross-section of the inhalation device 100 of FIGS. 15A-15B. As can be seen, the inhalation device 100 of FIGS. 15A-15B incorporates many of the features disclosed hereinabove for the various designs of inhalation devices 100, including, and not exhaustively: the liquid-retaining compartment 105 partially bounded on one side by a liquid-retaining wall 104: the definition of narrow sections as discussed with reference to FIG. 14: the distal casing 190 encompassing, at least circumferentially, the mesh membrane 185; and the internal power source 125 and electronic circuitry 135 connected by wire 146.

Referring to FIGS. 17A and 17B, the modular components of the inhalation device 100 of FIGS. 15A, 15B and 16 can be provided in a kit, packaged in a container 500. A first example of a kit is shown in FIG. 17A, comprising an inhalation-sensor section 460 that includes the distal portion 230, the neck portion 320, and the second proximal portion 210_{B}. The kit also includes a first proximal portion 210_{A}. In some embodiments, the kit of FIG. 17A can include, as shown, at least one additional first proximal portion 210_{A}. A second example of a kit is shown in FIG. 17B, comprising an inhalation-sensor section 450 that includes the distal portion 230 and the neck portion 320, and, unattached, the first and proximal portions 210_{A}, 210_{B}.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons skilled in the art to which the invention pertains.

It will be clear to the skilled artisan that any of the features described in connection with any of the figures can be combined with each other with the scope of the present invention even if not explicitly combined in this disclosure. For example, a design for which an airflow sensor was not explicitly shown may include an airflow sensor to trigger activation/initiation (or deactivation/cessation) of the piezo assembly and generation of mist by the mesh membrane, or a design for which a capillary pathway was not explicitly shown may include a capillary path for transport of liquid to the mesh membrane. As another non-limiting example, any of the designs illustrated can incorporate a liquid-retaining compartment effective to be filled using gravity and to retain liquid using a liquid-retaining wall, such that the mesh remains in contact with liquid, after the inhalation device is turned horizontal or ‘below horizontal’, i.e., with the container at least partly higher than the liquid-retaining compartment.

In the description and claims of the present disclosure, each of the verbs, “comprise”, “include” and “have”, and conjugates thereof, are used to indicate that the object or objects of the verb are not necessarily a complete listing of members, components, elements or parts of the subject or subjects of the verb. As used herein, the singular form “a”, “an” and “the” include plural references unless the context clearly dictates otherwise. For example, the term “a marking” or “at least one marking” may include a plurality of markings.

1. An electrically-powered inhalation device for delivery of an aerosol to the oropharynx of a user, the inhalation device comprising:
a. a distal portion including (i) an aerosol outlet defining a mist-exiting location and (ii) a piezo assembly including an ultrasonically vibrable mesh membrane, for producing, upon electrical activation, a mist comprising droplets of the liquid, the mesh membrane defining a mist-generating location; and
b. a neck portion including a narrow section, the narrow section being characterized by a minimum cross-sectional dimension that is at least 10% smaller than a minimum cross-sectional dimension of the distal portion passing through and parallel to the mesh membrane, at least a part of the narrow section being displaced proximally from the mesh membrane by at least 0.5 cm and not more than 6 cm,
wherein the inhalation device is shaped such that when the user's lips and/or teeth are transversely engaged with the narrow section, the mist-generating location resides distal to the user's teeth within the user's oral cavity and the mist-exiting location is in direct fluid communication with the user's oropharynx.

2. The inhalation device of claim 1, wherein the distal portion comprises a distal casing encompassing the mesh membrane at least circumferentially.

3. The inhalation device of either one of claim 1 or 2, wherein the at least a part of the narrow section is displaced proximally from the mesh membrane by at least 0.5 cm and not more than 5.5 cm, or by at least 0.5 cm and not more than 5 cm, or by at least 0.5 cm and not more than 4.5 cm, or by at least 0.5 cm and not more than 4 cm, or by at least 1 cm and not more than 6 cm, or by at least 1 cm and not more than 5.5 cm, or by at least 1 cm and not more than 5 cm, or by at least 1 cm and not more than 4.5 cm, or by at least 1 cm and not more than 4 cm.

4. The inhalation device of any preceding claim, wherein the narrow section is characterized by a minimum cross-sectional dimension that is at least 20% smaller than the minimum cross-sectional dimension of the distal portion passing through and parallel to the mesh membrane, or at least 30% smaller than the minimum cross-sectional dimension of the distal portion passing through and parallel to the mesh membrane, or at least 40% smaller than the minimum cross-sectional dimension of the distal portion passing through and parallel to the mesh membrane, or at least 50% smaller than the minimum cross-sectional dimension of the distal portion passing through and parallel to the mesh membrane.

5. The inhalation device of any preceding claim, wherein the minimum cross-sectional dimension of the narrow section and the minimum cross-sectional dimension of the distal portion define vectors that are coplanar, or within ±15° of being coplanar, or within ±30° of being coplanar, or within ±45° of being coplanar.

6. The inhalation device of any preceding claim, comprising a proximal portion that includes a power source for powering the piezo assembly.

7. The inhalation device of any preceding claim, comprising a proximal portion that includes a liquid inlet.

8. The inhalation device of any preceding claim, comprising a first proximal portion that includes a liquid inlet and a second proximal portion that includes a power source for powering the piezo assembly.

9. The inhalation device of either one of claim 7 or 8, wherein an outlet of the proximal portion that includes a liquid inlet is detachably attachable to the neck portion such that an interior volume of the proximal portion that includes a liquid inlet is arranged to be in fluid communication with an interior volume of the neck portion when a pressure-activated one-way valve is activated by pressure from the proximal portion that includes a liquid inlet.

10. The inhalation device of any preceding claim, wherein a center of gravity of the inhalation device is displaced proximally from a distal end of the narrow section when the inhalation device is in a liquid-empty state.

11. The inhalation device of any preceding claim, additionally comprising an inhalation sensor for monitoring a flow in an inhalation flow-path.

12. The inhalation device of claim 11, wherein the inhalation sensor is effective to detect an air pressure in the inhalation-flow path.

13. The inhalation device of claim 11, wherein the inhalation sensor is effective to detect a difference between an air pressure in the inhalation flow-path and an ambient air pressure outside the inhalation device.

14. The inhalation device of any one of claims 11 to 13, comprising control circuitry configured to initiate and/or cease activation of the mesh membrane in response to a result of the monitoring of the flow in the inhalation flow path.

15. The inhalation device of any preceding claim, wherein the distal portion comprises a liquid-retaining compartment in fluid communication with the neck portion, the liquid-retaining compartment being shaped to receive a quantity of the liquid from the neck portion by force of gravity when the inhalation device is in a first orientation, and to retain at least a part of the quantity against the force of gravity when the inhalation device is in a second orientation.

16. The inhalation device of claim 15, wherein the retaining is by a wall of the liquid-retaining compartment, the wall being effective to partially block an egress of the retained at least a part of the quantity.

17. The inhalation device of either one of claim 15 or 16, wherein the second orientation is such that substantially all of the mesh membrane is in liquid communication with the retained at least a part of the quantity.

18. The inhalation device of any one of claims 15 to 17, wherein the second orientation is such that a surface liquid level in the liquid-retaining compartment is higher than a surface liquid level in the container.

19. The inhalation device of any preceding claim, wherein the inhalation device is shaped such that when the user's lips and/or teeth are transversely engaged with the intermediate portion, the mist-generating location is at least 20% deep or at least 30% deep or at least 40% deep or at least 50% deep or at least 60% deep or at least 70% deep or at least 80% deep into an oral-cavity volume beneath the user's hard palate.

20. A kit comprising the inhalation device of any one of claims 8 to 19, packaged in a container such that the proximal portion that includes a liquid inlet is detached from the neck portion.

21. An electrically-powered inhalation device for delivery of an aerosol to the oropharynx of a user, the inhalation device comprising:
a. a distal portion including (i) an aerosol outlet defining a mist-exiting location and (ii) a piezo assembly including an ultrasonically vibrable mesh membrane, for producing, upon electrical activation, a mist comprising droplets of the liquid, the mesh membrane defining a mist-generating location; and
b. a neck portion including a narrow section, the narrow section being characterized by a minimum cross-sectional dimension that is at least 10% smaller than a minimum cross-sectional dimension passing through and parallel to the mesh membrane, a center of gravity of the inhalation device being displaced proximally from a distal end of the narrow section when the inhalation device is in a liquid-empty state,
wherein the inhalation device is shaped such that when the user's lips and/or teeth are transversely engaged with the narrow section, the mist-generating location resides within the user's oral cavity and the mist-exiting location is in direct fluid communication with the user's oropharynx.

22. The inhalation device of claim 21, wherein the distal portion comprises a distal casing encompassing the mesh membrane at least circumferentially.

23. The inhalation device of either one of claim 21 or 22, wherein the narrow section is characterized by a minimum cross-sectional dimension that is at least 20% smaller than the minimum cross-sectional dimension of the distal portion passing through and parallel to the mesh membrane, or at least 30% smaller than the minimum cross-sectional dimension of the distal portion passing through and parallel to the mesh membrane, or at least 40% smaller than the minimum cross-sectional dimension of the distal portion passing through and parallel to the mesh membrane, or at least 50% smaller than the minimum cross-sectional dimension of the distal portion passing through and parallel to the mesh membrane.

24. The inhalation device of any one of claims 21 to 23, wherein the minimum cross-sectional dimension of the narrow section and the minimum cross-sectional dimension of the distal portion define vectors that are coplanar, or within ±15° of being coplanar, or within ±30° of being coplanar, or within ±45° of being coplanar.

25. The inhalation device of any one of claims 21 to 24, comprising a proximal portion that includes a power source for powering the piezo assembly.

26. The inhalation device of any one of claims 21 to 25, comprising a proximal portion that includes a liquid inlet.

27. The inhalation device of any one of claims 21 to 26, comprising a first proximal portion that includes a liquid inlet and a second proximal portion that includes a power source for powering the piezo assembly.

28. The inhalation device of either one of claim 26 or 27, wherein an outlet of the proximal portion that includes a liquid inlet is detachably attachable to the neck portion such that an interior volume of the proximal portion that includes a liquid inlet is arranged to be in fluid communication with an interior volume of the neck portion when a pressure-activated one-way valve is activated by pressure from the proximal portion that includes a liquid inlet.

29. The inhalation device of any one of claims 21 to 28, wherein at least a part of the narrow section is displaced proximally from the mesh membrane by at least 0.5 cm and not more than 6 cm, or by at least 0.5 cm and not more than 5.5 cm, or by at least 0.5 cm and not more than 5 cm, or by at least 0.5 cm and not more than 4.5 cm, or by at least 0.5 cm and not more than 4 cm, or by at least 1 cm and not more than 6 cm, or by at least 1 cm and not more than 5.5 cm, or by at least 1 cm and not more than 5 cm, or by at least 1 cm and not more than 4.5 cm, or by at least 1 cm and not more than 4 cm.

30. The inhalation device of any one of claims 21 to 29, additionally comprising an inhalation sensor for monitoring a flow in an inhalation flow-path.

31. The inhalation device of claim 30, wherein the inhalation sensor is effective to detect an air pressure in the inhalation-flow path.

32. The inhalation device of claim 31, wherein the inhalation sensor is effective to detect a difference between an air pressure in the inhalation flow-path and an ambient air pressure outside the inhalation device.

33. The inhalation device of any one of claims 30 to 32, comprising control circuitry configured to initiate and/or cease activation of the mesh membrane in response to a result of the monitoring of the flow in the inhalation flow path.

34. The inhalation device of any one of claims 21 to 33, wherein the distal portion comprises a liquid-retaining compartment in fluid communication with the neck portion, the liquid-retaining compartment being shaped to receive a quantity of the liquid from the neck portion by force of gravity when the inhalation device is in a first orientation, and to retain at least a part of the quantity against the force of gravity when the inhalation device is in a second orientation.

35. The inhalation device of claim 34, wherein the retaining is by a wall of the liquid-retaining compartment, the wall being effective to partially block an egress of the retained at least a part of the quantity.

36. The inhalation device of either one of claim 34 or 35, wherein the second orientation is such that substantially all of the mesh membrane is in liquid communication with the retained at least a part of the quantity.

37. The inhalation device of any one of claims 34 to 36, wherein the second orientation is such that a surface liquid level in the liquid-retaining compartment is higher than a surface liquid level in the container.

38. The inhalation device of any one of claims 21 to 37, wherein the inhalation device is shaped such that when the user's lips and/or teeth are transversely engaged with the intermediate portion, the mist-generating location is at least 20% deep or at least 30% deep or at least 40% deep or at least 50% deep or at least 60% deep or at least 70% deep or at least 80% deep into an oral-cavity volume beneath the user's hard palate.

39. A kit comprising the inhalation device of any one of claims 27 to 38, packaged in a container such that the proximal portion that includes a liquid inlet is detached from the neck portion.
